(19) United States
(12) Patent Application Publication
(10) Pub. No.: US 2012/0215211 A1
(43) Pub. Date: Aug. 23, 2012

(54) DELIVERY FIBER FOR SURGICAL LASER TREATMENT AND METHOD FOR MAKING SAME

(75) Inventor: Richard P. TUMMINELLI, Ashland, MA (US)

(73) Assignee: Coherent, Inc., Santa Clara, CA (US)

(21) Appl. No.: 13/107,585

(22) Filed: May 13, 2011

Related U.S. Application Data
(60) Provisional application No. 61/444,010, filed on Feb. 17, 2011.

(51) Int. Cl. A61B 18/18; B32B 7/02; G02B 6/26

(52) U.S. Cl. 606/16; 385/31; 428/212

(57) ABSTRACT

An optical surgical fiber assembly for delivering laser radiation from a laser radiation source to a treatment site has a sealed off capillary enclosing a delivery end of the fiber. The capillary is formed from an outermost layer of fused silica and an adjacent layer of boron-doped fused silica having a higher CTE than that of the fused silica. The capillary is shrink-fitted onto the delivery end of the fiber. A compressive stress is imparted to the outermost layer of the capillary as a result of the shrink-fitting process and the CTE difference between the layers. This provides mechanical hardening of the surface of the outermost layer.

### PRIORITY

This application claims priority to U.S. Provisional Application Ser. No. 61/444,010, filed Feb. 17, 2011, the disclosure of which is incorporated herein by reference.

### TECHNICAL FIELD OF THE INVENTION

The present invention relates in general to fiber capillary assemblies used in laser surgical procedures. The invention relates in particular to methods for improving damage resistance and lifetime of side-firing fiber capillary assemblies used in laser surgical procedures.

### DISCUSSION OF BACKGROUND ART

In laser surgical procedures where radiation from a laser is delivered to a treatment site by an optical fiber, a common practice is to use a sealed-off capillary encasing the distal (delivery) end of a fiber for protecting the distal end of the fiber from damage by several mechanisms. Such a capillary is particularly useful in a delivery fiber designed to deliver radiation laterally from the fiber in a manner usually referred to in the art as “side-firing,” for reasons discussed further below.

FIG. 1 schematically illustrates a prior-art surgical delivery device 10 including an optical fiber 12 and a sealed-off capillary 26. Optical fiber 12 comprises a core 14 surrounded by a cladding 16 that constrains laser radiation 18 coupled into the fiber at the proximal (input) end 12A thereof to propagate along the core.

Device 10 is designed for side-firing. Distal end 12B of the fiber is cut or polished (beveled) to form a surface 28 at an angle to the length direction of the fiber. Sealed-off capillary 26 is sealed to fiber 12 by a weld 20 and is arranged to leave a space 24 surrounding angled surface 28 of the optical fiber. Angled surface 28 of optical fiber 12 is ideally made highly reflective for the laser radiation propagating along core 14 of the fiber such that radiation 18 is directed by the angled surface laterally through cladding 16 and through capillary 26 at an external region 22 on the capillary. High reflectivity of angled surface 28 can be provided by a reflective coating on the angled surface, or, preferably, by relying on total internal reflection (TIR) at the angled surface. Reflective coatings may be subject to laser damage dependent, inter alia, on the laser power, materials of the coatings, and method of deposition of the coatings. The sealed space surrounding the angled surface ensures that TIR can be relied on even when the fiber is immersed in a fluid.

In the assembly of FIG. 2, emergent laser radiation 18 can be utilized for surgical procedures. One such procedure is Holmium Laser Enucleation of the Prostate (HoLEP). In this procedure, a holmium:YAG (Ho:YAG) laser is used to remove obstructive prostate tissue. The Ho:YAG surgical laser is a solid-state, pulsed laser that emits radiation at a wavelength of approximately 2100 nanometers (nm). For this actual surgical procedure, surgical fiber device 10 is utilized with the laser source, together with other instruments and devices (not shown). During a procedure, water and other materials can come into contact with surgical fiber device 10, particularly with capillary 26 thereof, and potentially cause damage to surgical fiber device.

During experiments to test a prior-art fiber assembly 10 for a HoLEP procedure, the outside of capillary 26 at external, (radiation-exit) position 22 became pitted (damaged) over a period of use. It is believed that this pitting could be caused by one or more damage mechanisms, including, inter alia, back-reflection of laser radiation 18, water-vapor bubbles, or ablated material depositing on the capillary.

The pitting of the capillary's external (radiation-exit) position 22 initially led to reduced efficiency of the amount of laser radiation delivered from the capillary, which, at a minimum would require additional time to perform the required surgical procedure. Eventually the pitting could be sufficient to scatter laser radiation in a manner such that the surgical procedure could not be performed. It was estimated that this point could be reached before a HoLEP procedure could be completed. This would require that a replacement device be used to complete the procedure, which could increase the duration and cost of the procedure.

It is believed that the above discussed laser-induced pitting could be at least mitigated by mechanically hardening the outer surface of the capillary, specifically, by creating a relatively high compressive stress in the surface. Inducing compressive stress in glass and silica surfaces has been reported to improve laser damage resistance of those surfaces. Certainly, silica and glass surfaces have been mechanically hardened by inducing compressive stress therein.

Well-known methods of stress hardening glass surfaces include heat tempering and chemical tempering. It is believed, however, that these procedures are not applicable to hardening the surface of a capillary such as above-described capillary 26, for various reasons. Accordingly, there is a need for another compressive-stress-inducing method which can stress-harden the surface of the capillary, with the potential of improved resistance to laser-induced degradation of the capillary during laser surgical procedures.

### SUMMARY OF THE INVENTION

The present invention is directed to optical apparatus for delivering laser radiation to a treatment site in laser surgical procedures. In one aspect of the invention, the apparatus comprises an optical fiber having a core surrounded by a cladding. The optical fiber has a proximal end into which the laser radiation is input, and a distal end from which the laser radiation is delivered after propagating along the fiber. The distal end of the optical fiber is surrounded by a closed-end capillary arranged such that there is a space between the distal end of the fiber and the closed end of the capillary. The capillary is sealed to the cladding. The capillary includes first and second layers where the first layer is an outermost layer of the capillary and the second layer is adjacent to the first layer. The first layer has a coefficient of thermal expansion (CTE) different from that of the second layer, and the CTE of the second layer is higher than that of the first layer. The capillary is formed in a manner such that the first layer is under compressive stress as a result of the difference in CTE between the first and second layers.

In another aspect of the invention, the capillary is formed from capillary tubing drawn from a preform. In one preferred embodiment, the preform comprises a cylinder including first and second layers, the first layer being an outermost layer of the cylinder, and the second layer being adjacent the first layer. The second layer has a higher coefficient of thermal expansion (CTE) than that of the first layer.

In one example of the preform, the first layer of the preform cylinder is a tube of fused silica having a CTE of about 0.5×10⁻⁶/° K. The second layer of the cylinder is a layer of boron-oxide doped fused silica having a CTE of about 2×10⁻⁶/° K.

### BRIEF DESCRIPTION OF THE DRAWINGS

The accompanying drawings, which are incorporated in and constitute a part of the specification, schematically illustrate a preferred embodiment of the present invention, and together with the general description given above and the detailed description of the preferred embodiment given below, serve to explain principles of the present invention.

FIG. 1 is an un-shaded cross-section view schematically illustrating a prior-art surgical side-firing fiber arrangement including an optical fiber, a distal end of which is surrounded by a sealed-off capillary.

FIG. 2 is an un-shaded cross-section view schematically illustrating a preferred embodiment of a side-firing fiber arrangement in accordance with the present invention similar to the arrangement of FIG. 1, but wherein the sealed off capillary has inner and outer layers, with the inner layer having a higher coefficient of thermal expansion than that of the outer layer, and wherein the capillary is formed by a method which creates a high compressive stress in the outer layer.

FIG. 3 is three-dimensional view schematically illustrating a pre-form structure in accordance with the present invention being drawn into capillary tubing used for forming the two-layer, sealed-off capillary of FIG. 2.

FIG. 4A, FIG. 4B, FIG. 4C schematically illustrate stages in the formation of the two-layer sealed-off capillary of FIG. 2 from the capillary tubing of FIG. 3.

### DETAILED DESCRIPTION OF THE INVENTION

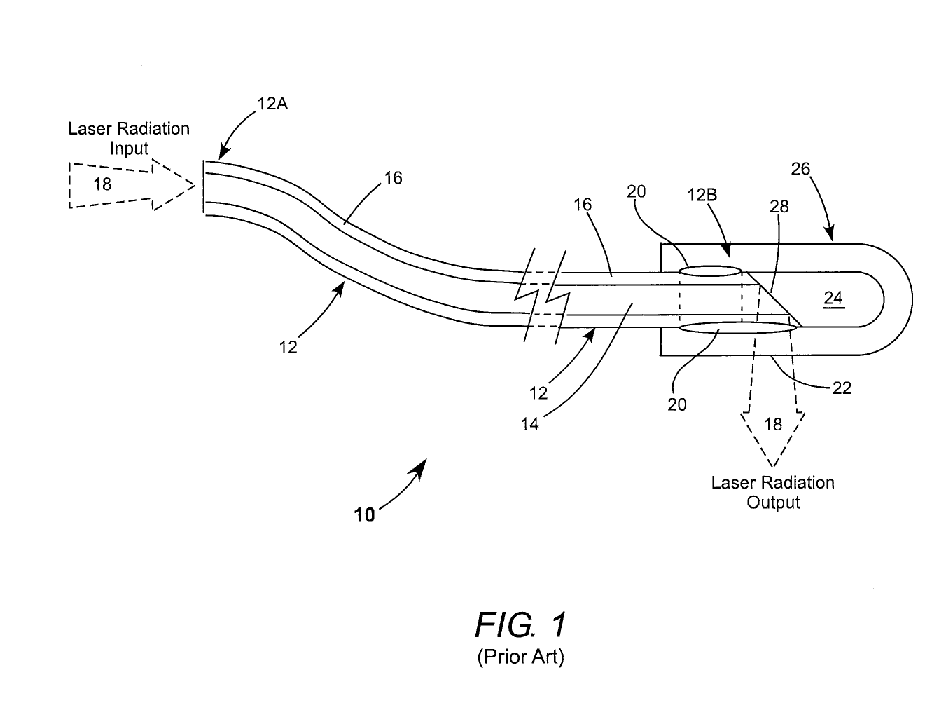

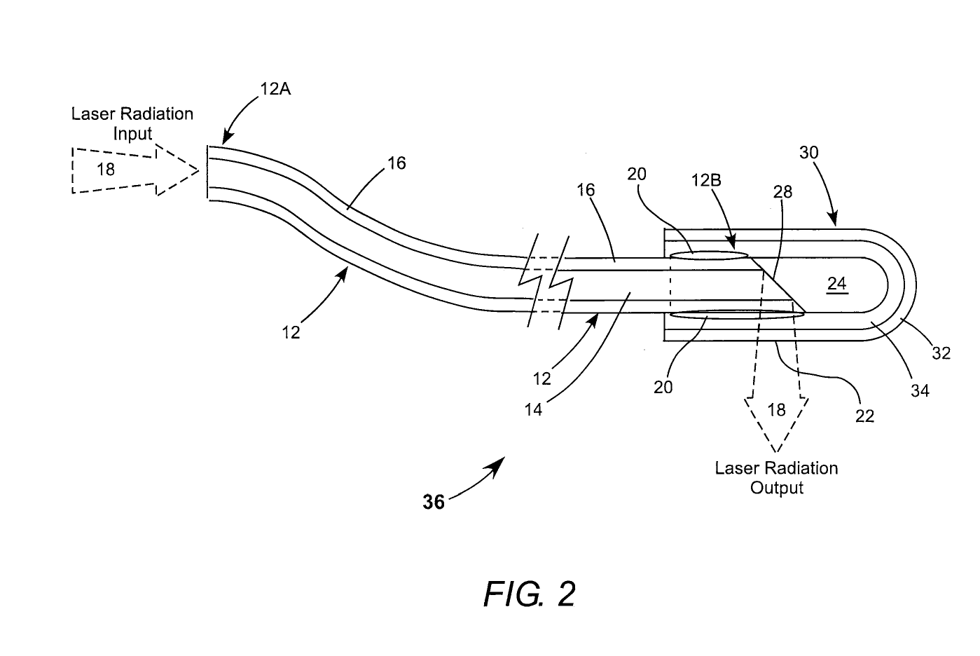

Referring now to the drawings, wherein like components are designated by like reference numerals, FIG. 2 schematically illustrates a preferred embodiment 36 of a surgical delivery fiber arrangement in accordance with the present invention. This arrangement is similar to prior-art arrangement 10 of FIG. 1 with an important exception that prior-art capillary 26 of arrangement 10 is replaced in inventive arrangement 36 by a sealed-off (closed-end) capillary 30 arrangement including an outermost layer 32 and an adjacent layer 34.

Outermost layer 32 has a coefficient of thermal expansion (CTE) significantly lower than that of adjacent layer 34. Capillary 30 is formed in a manner such that outermost layer 32 is under compressive stress as a result of the difference in CTE between the outermost and adjacent layers, and because of a preferred process of sealing capillary 30 to fiber 12 at seal 20. In general it is preferred that layer 34 has a thickness at least about twice that of layer 32, and that layer 34 has a CTE at least about twice that of layer 32.

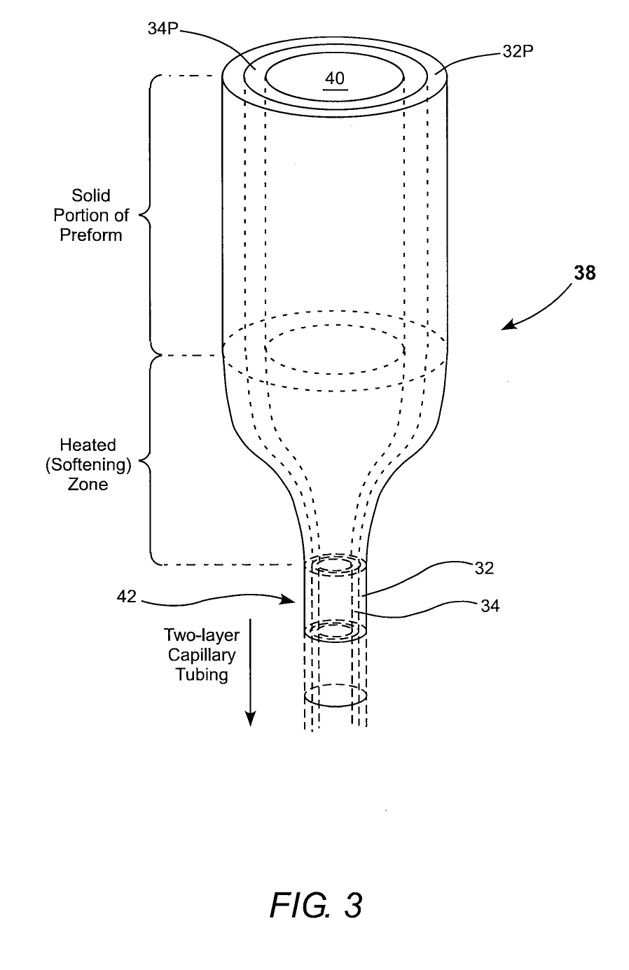

In one example of capillary 30, outermost layer 32 is a layer of fused silica having a thickness of about 0.01 millimetres (mm) and a CTE of about 0.5×10⁻⁶/° K. Adjacent layer 34 is a layer of boron-doped (boron-oxide-doped) fused silica having a thickness of about 0.45 mm and a CTE of about 2×10⁻⁶/° K. Space 24 has a diameter of about 0.4 mm. A description of a preferred method of forming closed-end capillary 30 is set forth below, beginning with reference to FIG. 3.

Here, a preform structure 38 includes an outermost layer 32P and an adjacent layer 34P. The suffix “P” on the reference numerals is added to indicate that those layers become layers 32 and 34 in finished capillary 30. In a preferred example, layer 32P is a fused-silica tube (cylinder) having an outside diameter (OD) of about 16.65 mm and a wall-thickness of about 1.1 mm. Layer 34P is a layer of boron-oxide doped (boron-doped) fused silica having a wall thickness of about 4.725 mm, leaving a hollow interior 40 of the preform having an diameter of about 5.0 mm. Layer 34P is preferably formed on the inside wall of tube 32P by modified chemical vapor deposition (MCVD). Boron oxide (B₂O₃) doping of the fused silica increases the CTE of fused silica and lowers the refractive index. A preferred boron oxide doping percentage is about 20.0 Mole %. This provides about the above-exemplified CTE of the boron-oxide-doped fused silica.

A lower part of the preform is heated above the softening point of the preform materials and is drawn, then solidifies, to form two-layer capillary tubing 42. The inside diameter of the tubing is selected to be about 20 micrometers (μm) greater than the outside diameter of fiber 12 on which finished capillary 30 of FIG. 2 is to be formed. At this stage, layer 32 of the capillary tubing will have a high compressive stress as a result of the CTE difference between layers 32 and 34 during solidification.

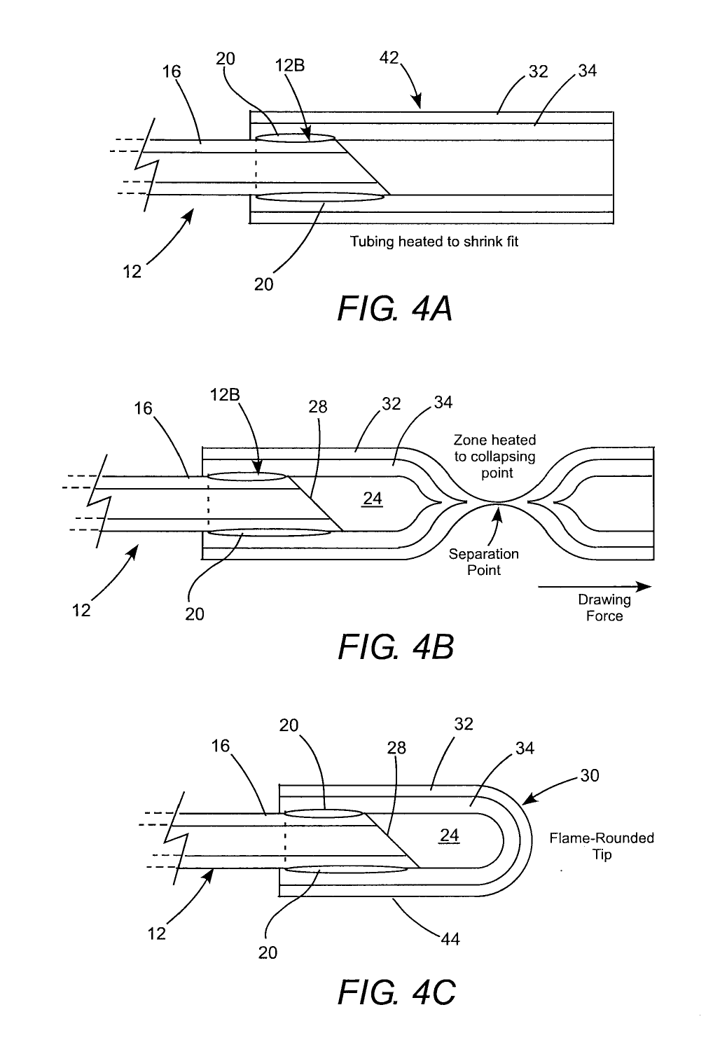

FIG. 4A, FIG. 4B, and FIG. 4C schematically illustrate stages in the formation of capillary 30 in the fiber assembly of FIG. 2. First (see FIG. 4A), distal end 12B of fiber 12 is inserted into a length of capillary tubing 42, which is heated in the region of overlap with the fiber to a temperature at which the capillary tubing softens to a point, which causes the capillary tubing to shrink onto the distal end of the fiber, thereby forming a shrink-fit seal or weld in region 20 around the distal end of the fiber. The heat is removed and the capillary tubing solidifies. During the softening of the length of capillary tubing, compressive stress originally in outer layer 32 of the capillary tubing is substantially annealed out, but reappears as a result of solidification after the shrink-fitting. This is the principal source of the compressive stress in outermost layer 32.

Referring next to FIG. 4B, following the shrink-fitting of the capillary tubing onto distal end 12B of fiber 12, a zone of the capillary tubing is heated to a collapsing point, while and end of the tubing is drawn away from the fiber. This causes the capillary tubing to collapse on itself, and separate in the heated zone to seal-off (close the end of) that capillary tubing attached to the fiber forming sealed-off space 24. The separated end can then be rounded off by flame-heating, as depicted in FIG. 4C, to form finished capillary 30 on fiber 12, with outermost layer 32 of the capillary under the desired high compressive stress.

Computer modeling was used to analyze and model the stress of the aforementioned inventive capillary and fiber. Commercially-available, finite element analysis (FEA) software was used to calculate the stresses in the inventive capillary and fiber. For this modeling, the input parameters were the above discussed materials and exemplary dimensions of the capillary and assumed initial relaxed and final stressed temperatures of 1700° C. and 25° C., respectively. The CTE and Young's modulus of the capillary and fiber materials were assumed to be constant through this temperature range. The FEA software calculated the axial stress of the capillary at the outside surface 44 of outmost layer 32 of inventive capillary 30 (FIG. 4C) to be approximately −100 gigapascals (GPa) (compressive stress) along the length of the capillary.

What is claimed is:

1. Optical apparatus for delivering laser radiation to a treatment site in laser surgical procedures, the apparatus comprising:
an optical fiber having a core surrounded by a cladding, the optical fiber having a proximal end into which the laser radiation is input and a distal end from which the laser radiation is delivered after propagating along the fiber;
the distal end of the optical fiber being surrounded by a closed-end capillary arranged such that there is a space between the distal end of the fiber and the closed end of the capillary, and the capillary being sealed to the cladding of the fiber; and
wherein the capillary includes first and second layers, the first layer being an outermost layer of the capillary and the second layer being adjacent the first layer, the CTE of the second layer being higher than that of the first layer, and the capillary being formed in a manner such that the first layer is under compressive stress as a result of the difference in CTE between the first and second layers.

2. The apparatus of claim 1, wherein the first layer of the capillary is a fused silica layer.

3. The apparatus of claim 1, wherein the second layer of the capillary is a boron-oxide doped layer.

4. The apparatus of claim 1, wherein the distal end of the optical fiber has a surface at an angle to the length of the fiber arranged such that that radiation propagating along the fiber is reflected by the surface laterally out of the fiber.

5. The apparatus of claim 1, wherein the second layer has a thickness at least about twice that of the first layer and has a CTE at least about twice that of the first layer

6. The apparatus of claim 5, wherein the first layer of the capillary has coefficient of thermal expansion (CTE) of about 0.5×10⁻⁶/K, and the second layer of the capillary has CTE of about 2×10⁻⁶/K.

7. The apparatus of claim 6 wherein the first layer has a thickness of about 0.1 millimeters and the second layer has a thickness of about 0.45 millimeters.

8. A preform structure for drawing capillary tubing, the preform structure comprising:
a cylinder including first and second layers, the first layer being an outermost layer of the cylinder and the second layer being adjacent the first layer, the second layer having a higher coefficient of thermal expansion (CTE) than that of the first layer.

9. The preform structure of claim 8, wherein the first layer is a fused-silica tube having a CTE of about 0.5×10⁻⁶/° K.

10. The preform structure of claim 9, wherein the first layer is a fused-silica tube having a CTE of about 2×10⁻⁶/° K.

11. A side firing optical fiber comprising:
an optical fiber having an input end and an exit end, said fiber having a core and an outer cladding, said exit end terminating in a tip have a planar end face oriented at an angle to the longitudinal axis of the fiber; and
a transparent end cap affixed to the exit end of the fiber, said end cap having an open region adjacent the tip, said end cap including region extending from the inner surface thereof that is in compressive stress.

12. A fiber as recited in claim 11 wherein the end cap is formed from fused silica and the region in compressive stress is doped with boron.

13. A fiber as recited in claim 11 wherein the level of boron doping is sufficient so the CTE of the region is at least about twice the CTE of the outer region of the end cap.

14. A fiber as recited in claim 13 wherein the region that is in compressive stress has a thickness greater than thickness of the remaining portion of the cap.
